(19) United States
(12) Patent Application Publication
(10) Pub. No.: US 2014/0275259 A1
(43) Pub. Date: Sep. 18, 2014

(54) Direct Lipid to Membrane Drug Delivery

(71) Applicant: APPLIED BIORESEARCH, INC., Draper, UT (US)

(72) Inventors: James F. Palmer, Newport Beach, CA (US); Robert A. Settineri, Irvine, CA (US)

(73) Assignee: APPLIED BIORESEARCH, INC., Draper, UT (US)

(21) Appl. No.: 14/207,424

(22) Filed: Mar. 12, 2014

Related U.S. Application Data
(60) Provisional application No. 61/778,256, filed on Mar. 12, 2013.

(51) Int. Cl. A61K 31/198

(52) U.S. Cl. CPC A61K 31/198
USPC 514/566

(57) ABSTRACT

Described herein is a method of delivering hydrophilic drugs directly from a hydrophobic carrier and/or excipient to absorbing cell membranes. More specifically, CaNa₂ EDTA is delivered rectally from a suppository containing methylbutylketone in a carrier as an excipient.

This application claims benefit of U.S. Provisional Application 61/778,256 filed Mar. 12, 2013

### FIELD OF THE INVENTION

This invention is directed to compositions for providing an excipient delivery system for negatively-charged drugs, particularly calcium disodium ethylenediaminetetraacetate (CaNa₂EDTA) to enhance absorption of the drug through living cell membranes. Negatively charged (anionic) drugs are poorly absorbed across cell membranes if they become solvated in water, such as body fluids including, but not limited to, saliva, gastric juices, colonic fluid, nasal, vaginal, mucosal, interstitial fluids and rectal fluid. Because anionic drugs are less readily or not adequately dissolved in these fluids, these drugs are commonly administered by intravenous drip, intraperitoneal injections, intranasal instillation, inhalation therapy or subcutaneous injection. These methods are invasive and painful, and can be expensive.

### BACKGROUND

Drug absorption is typically predicted based on tests designed to evaluate oral administration. There are several distinct steps in modeling or understanding drug absorption including release from the excipient; solvation in the body fluid; absorption by the luminal or mucosal cells and distribution to local or systemic sites. (Shono Y, Jantratid E, Kesisoglou F, Reppas C, Dressman JB. “Forecasting in vivo oral absorption and food effect of micronized and nanosized aprepitant formulations in humans.” Eur J Pharm Biopharm. pp 95-104 Sep;76(1),(2010))

The first step is the release of the drug from the excipient or carrier. This is universally tested by dissolution. The drug and excipient combination are chosen to dissolve in the target body fluid. For instance, a sublingual delivery would be optimized to dissolve in saliva, while a stomach delivery would be designed to be released in the acidic milieu of the gastric juices. Enteric coatings that are designed to resist dissolution in an acidic environment are used to deliver drugs to the small intestine. Suppositories are designed to dissolve in the rectal fluid (or to melt, for example from hydrophobic carriers such as cocoa butter and MBK in a fatty acid base).

Oral delivery systems include tablets, gel capsules and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g. propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

### SUMMARY

MBK, and more particularly MBK in a fatty acid base, is a preferred excipient for thorough and consistent release of CaNa₂ EDTA from a rectal suppository. The CaNa₂ EDTA was dissolved or suspended in the delivery means, and the permeability of the drug was tested for the appropriate target. Cells in the digestive track, such as CACO-2 cells are typically used to model absorption in the small intestine. CACO-2 cells, developed by the Sloan-Kettering Institute from human epithelial colorectal adenocarcinoma cells, are widely used to predict drug absorption. Parallel absorption membrane permeability assay (PAMPA) uses an artificial cell membrane to model drug permeability. In either test, the drug is dissolved in an aqueous solution mimicking the target gastric fluid. The fluid is contacted with the membrane, and the passage of the drug through the membrane over a period of time is used to assess permeability.

### DETAILED DISCUSSION

A particular anionic drug, namely EDTA and more particularly calcium disodium ethylenediaminetetraacetate (CaNa₂EDTA), is FDA-approved for treatment of lead poisoning via intravenous administration and the utility for this purpose this use has been recently reconfirmed. (Born T, Kontoghiorghe C N, Spyrou A, Kolnagou A, Kontoghiorghes G J. “EDTA chelation reappraisal following new clinical trials and regular use in millions of patients: review of preliminary findings and risk/benefit assessment.”, Toxicol Mech Methods. Jan;23(1):11-7(2013). However, numerous attempts to provide more convenient oral delivery have failed.

It has been found by applicants from animal studies that delivery of EDTA via suppository using methylbutylketone (MBK), also known as 2 hexanone, a hydrophilic compound in a hydrophobic fatty acid base is successful. Administration via suppository of CaNa₂EDTA with MBK as the excipient (see Table 1) allowed over 36.3% of the drug to be absorbed within eight hours in a rat pharmacokinetic model. Table 1 is data showing bioavailability of CaNa₂EDTA delivered from a suppository using a rat animal model compared to IV delivery. However, prior studies using an aqueous solution have shown that a much lower absorption is found in dogs (˜13%,) and a highly variable and lesser absorption was noted in humans when the EDTA is administered rectally. (Rabau M Y, Baratz M, Rozen P, “Na₂ ethylenediaminetetraacetic acid retention enema in dogs. Biochemical and histological response”. Gen Pharmacol.; 22(2):329-30. (1991))

TABLE 1
Bioavailabiity and Pharmacokinetics of CaNa₂EDTA Rectal Suppository vs IV Administration

| | | | | | AUC | | | | Absolute |
|---|---|---|---|---|---|---|---|---|---|
| | | | Dose | AUC | Inf | Halflife | Cmax | Tmax | Bioavailability |
| Group | Route | Stat. | (mg/kg) | (μg × Hr/mL) | (μg × Hr/mL) | (Hour) | (μg/ml) | (Hour) | (%) |
| B | Intravenous | MEAN | 1.37 | 1.86 | 1.91 | 1.50 | 2.07 | 0.083 | N/A |
| | | SD | 0.06 | 0.20 | 0.19 | 0.34 | 0.35 | 0.000 | |
| | | N | 4 | 4 | 4 | 4 | 4 | 4 | |
| C | Rectal | MEAN | 213.5 | 105.8 | 307.3 | 8.20¹ | 30.6 | 0.417 | 36.3 |
| | | SD | 12.0 | 32.2 | 225.6 | 5.61 | 10.6 | 0.144 | |
| | | N | 3 | 3 | 3 | 3 | 3 | 3 | |

Absolute bioavailability (%) = (AUCₜₑₛₜ × Dose_{ref}) × 100
(AUC_{ref} × Doseₜₑₛₜ)
Where “test” data is the rectal data, and “ref” (reference) data is the intravenous data.
¹The terminal elimination phase was not observed, therefore, this calculation is interpolated and longer sample intervals should be examined.

Likewise, orally administered enteric-coated EDTA, which provides EDTA to the higher pH environment in the small intestine, is not well absorbed. Enteric coatings, which consist, for example, of polymers that are resistant to dissolving in stomach acid but release the drug in the higher pH of the small intestine or colon can be provided in an attempt to avoid the acidic of the small intestine. Hall E J, Batt R M, Brown A., “Assessment of canine intestinal permeability, using 51 Cr-labeled ethylenediaminetetraacetate.” Am J Vet Res. Dec;50(12):2069-74 (1989)

An explanation for the poor gastric absorption of EDTA can be the behavior of calcium disodium EDTA or disodium EDTA solvated in water at different pHs. EDTA has six different pKas, causing it to be strongly ionic and thus highly water soluble at higher pHs.(Coleman, William F. “Molecular Models of EDTA and Other Chelating Agents”, (Journal of Chemical Education, Vol. 85 No. 9 September (2008)

However, at lower pHs, EDTA becomes only very sparingly soluble. It is believed that this causes orally ingested EDTA to precipitate in stomach acid and is thus lost from further bioavailability. Enteric coating will protect the EDTA from stomach acid, but the solvation of the highly hydrophilic drug in the gastric fluid will still cause it to be resistant to absorption.

In the second stage of modeling drug absorption, PAMPA tests were run at pH 7.0 and pH 9.6 in anticipation of optimizing a suppository delivery system for CaNa₂ EDTA. PAMPA measures how quickly the drug can exit a water-solvated environment and pass through a cell membrane. Since most drugs are hydrophobic, it is expected that there will be significant absorption by the cells lining the digestive tract.

However, it was suspected that the PAMPA tests might be negative for CaNa₂EDTA in an aqueous composition containing MBK based on the highly hydrophilic nature of the drug. The results of those PAMPA tests were indeed completely negative—no drug was found to have passed through the artificial cell membrane after 5 hours of testing. Test results were validated by use of low and high control drugs Atenolol and Verapamil.

The possibility that a protein amino acid transporter, hPAT1, was responsible for higher-than-expected absorption rate with the suppository route of delivery was then evaluated. However, studies have shown that while the hPAT1 transporter is readily found in the small intestine, it is not found in particular abundance in the colon or rectum. (Broberg M I, Holm R, Tønsberg H, Frølund S, Ewon K B, Nielsen A l, Brodin B, Jensen A, Kall M A, Christensen K V, Nielsen C U “Function and expression of the proton-coupled amino acid transporter PATI along the rat gastrointestinal tract: implications for intestinal absorption of gaboxadol.” Br J Pharmacol. Oct;167(3):654-65 (2012))

The conclusion was reached that it was possible that the high rate of bioavailability observed in the rat study was due to the direct transfer of CaNa₂EDTA from the hydrophobic MBK excipient to the mucosal cells in the rectum.

It is known that cell permeability for small molecular weight drugs is highest for cationic drugs and decreases as the drug is more neutral and there is lesser amount of transport for negatively charged drugs. It is further known that hydrophilic drugs are more easily absorbed than hydrophobic drugs. Since CaNa₂EDTA is both anionic and hydrophilic it is therefore postulated that the mechanism for increasing absorption was by direct transfer from the hydrophobic MBK excipient to the mucosal cells.

To prepare the suppository the CaNa₂EDTA in its crystalline form was mixed with an equal weight of MBK in a fatty acid base or melted cocoa butter at about 50° C., the MBK component in the fatty acid base or cocoa butter as excipients are non-ionic, or uncharged (not negatively charged). Other inert solids, such as Methocel® (methylcellulose and hydroxypropyl methylcellulose polymers) may be included. Sized for human use, the composition was then formed into a rectal suppository with a typical size containing 50% of the MBK component and about 50% of CaNa₂EDTA. In a preferred embodiment the suppository contains about 600 milligrams of MBK component and about 600 milligrams of CaNa₂EDTA .In studies with four test subjects the results were as follows:

| | | TOTAL mg |
|---|---|---|
| | SAMPLE | 0-24 HRS |
| PCCA Base MBK ™, | 1 | 8.63 |
| | 2 | 0.5 |
| | 3 | 6.25 |
| | 4 | 3.63 |
| AVG mg | | 4.75 |
| % of ADMINISTERED | | 0.6% |
| RSD | | 74% |

A typical human rectal suppository is a conical or torpedo shaped item about 2-3 centimeters in length. Suppositories for adults weigh about 2-3 grams each; children suppositories weigh about 1-2 grams each. The typical carriers used are waxy materials in which the active ingredients have been dissolved or suspended. The carrier can comprise, but is not limited to, glycerin, glyceryl monopalmitate, glyceryl monostearate, hydrogenated coconut oil fatty acids, cocoa butter, and hydrogenated palm kernel oil fatty acids.

The same result could be achieved by allowing the drug to dissolve in the body fluid and adjusting the pH. However, this could cause precipitation and loss of bioavailability as discussed above. As a result, the direct transfer of the non-ionic drug from the excipient-containing suppository to the absorbing cells allows the correct and advantageous optimization and delivery of hydrophilic anionic drugs.

The above process bypasses the traditional concept of solvation of the drug into the surrounding body fluid and is dependent on the contact of the CaNa₂EDTA-MBK containing composition directly with the absorbing cells. Mass transfer of the drug through the excipient to the cell membrane is assumed to occur through diffusion in light of the small volume of rectal fluid present (about 3 ml with a pH of about 7.5) compared to the volume of a typical suppository (also about 3 ml for adults).

This composition disclosed herein can be used to better define molecular models; better design and choose excipients that interact with absorbing cells and exclude water; and re-design PAMPA and CACO-2 analyses to test permeability of a drug in a hydrophobic medium versus an aqueous medium. Other dosage forms that will benefit from this new understanding of hydrophilic drug absorbance are identified herein.

In addition, solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents other than EDTA.

We claim:

1. A suppository for use in chelating heavy metals in a living being comprising:
i. calcium disodium ethylenediaminetetraacetate (CaNa₂ EDTA),
ii. a hydrophobic excipient, and
iii. a carrier comprising one or more of glycerin, glyceryl monopalmitate, glyceryl monostearate, hydrogenated coconut oil fatty acids, cocoa butter, and fatty acids.

2. The suppository of claim 1 wherein the hydrophobic excipient is methylbutylketone.

3. The suppository of claim 2 comprising about 50% of a MBK containing component and about 50% of CaNa₂EDTA.

4. The suppository of claim 2 comprising about 600 mg of a MBK containing component and about 600 mg of CaNa₂EDTA.

5. The suppository of claim 3 being about 2-3 centimeters in length and having suppositories weigh about 1-2 grams each.
